(19) United States
(12) Patent Application Publication
(10) Pub. No.: US 2010/0263061 A1
(43) Pub. Date: Oct. 14, 2010

(54) Properdin Modulation of Alternative Pathway and Uses Thereof

(75) Inventor: Wenchao Song, Rosemont, PA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: The Trustees of the University of Pennsylvania

(21) Appl. No.: 12/663,690

(22) PCT Filed: Jun. 11, 2008
(86) PCT No.: PCT/US08/07270

§ 371 Date: Jun. 8, 2010

Related U.S. Application Data
(60) Provisional application No. 60/929,066, filed on Jun. 11, 2007.

(51) Int. Cl. A61K 39/395; A01K 67/027; C12N 5/10; A61K 49/00; C07K 16/18; C12N 15/09; A61P 31/00

(52) U.S. Cl. 800/3; 424/158.1; 800/14; 800/18; 435/325; 530/389.3; 800/25

(57) ABSTRACT

This invention relates to selective activation of the alternative pathway (AP) using anti-Properdin antibodies. Specifically, the invention relates to methods for treating an AP complement-mediated pathology or AP mediated condition in a subject by contacting the subject with an anti-Properdin antibodies. Likewise, properdin knockout transgenic non-human mammals and their use are provided.

### GOVERNMENT INTEREST

This invention was supported, in part, by National Institutes of Health grants AI-62388, AI-49344, AI-44970. The government may have certain rights in the invention.

### FIELD OF INVENTION

This invention is directed to selective activation of the alternative pathway (AP) using anti-Properdin antibodies. Specifically, the invention is directed to methods for treating an AP complement-mediated pathology or AP mediated condition in a subject by contacting the subject with an anti-Properdin antibodies. Likewise, properdin knockout transgenic non-human mammals and their use are provided.

### BACKGROUND OF THE INVENTION

The complement system provides a first line of host defense against invading pathogens. Activation of complement occurs via 3 different pathways, the classical, lectin and alternative pathway. The classical pathway is initiated by antigen-antibody binding. The lectin pathway is triggered when mannose-binding lectins (MBL) interact with surface sugar molecules on microorganisms. Activation of both pathways leads to the assembly of the classical pathway C3 convertase C4b2a, although direct cleavage of C3 by MBL-associated serine proteases can also occur. The alternative pathway (AP) is a self-amplification loop driven by the AP C3 convertase, C3bBb. AP activation can occur secondary to classical or lectin pathway activation, or is initiated independently. In the latter case, a low level spontaneous C3 ‘tick-over’ generates the initial C3bBb, which rapidly propagates AP in the absence of adequate regulation. Thus, AP complement activation on non-self surfaces with no or insufficient negative regulation is considered a default process, whereas autologous cells typically avoid this outcome with the help of multiple membrane-bound and fluid phase complement inhibitory proteins.

In contrast to the existence of numerous inhibitory proteins, the plasma protein properdin is the only known positive regulator of the complement activation cascade. Discovered more than 50 years ago, properdin was at first regarded as an initiator of the AP complement, acting in a manner that was analogous to antibodies of the classical pathway. The existence of properdin and AP, known at one time as the ‘properdin pathway’ was not immediately accepted and became a subject of debate. While the importance of AP in complement activation has since been validated and is now textbook knowledge, the concept that properdin is the driving force of AP activation was essentially abandoned, to be replaced by the currently held view that properdin facilitates AP complement activation by extending the half-life of the nascent C3bBb convertase. Recently, it was demonstrated that surface C3b-bound properdin could serve as a platform for new C3bBb assembly.

This pointed out a more complex mechanism of action of properdin in AP complement activation and brought afore the need for further investigation on properdin function and its use in imparting immunity to properdin-deficient individuals.

### SUMMARY OF THE INVENTION

In one embodiment, the invention provides method of treating an AP complement-mediated pathology in a subject, comprising the step of administering to said subject an is alternative-pathway-specific anti-Properdin antibody, thereby inhibiting the generation of a C3bBb protein.

In another embodiment, the invention provides a method of inhibiting properdin-dependent, microbial antigen-, non-biological foreign surface- or altered self tissue-triggered AP complement activation in a subject, comprising the step of administering to said subject an alternative-pathway-specific, anti-Properdin antibody, thereby inhibiting the generation of a C3bBb protein.

In one embodiment, the invention provides a transgenic non-human mammal and progeny thereof whose genome comprises a disruption of a Properdin-encoding gene such that the mammal lacks or has reduced levels of functional Properdin.

In another embodiment, the invention provides A method for identifying in vivo a biological activity of a compound, said method comprising the steps of: providing a transgenic non-human mammal incapable of expressing properdin; administering said compound to said non-human mammal; determining an expressed pathology of said non-human mammal; and identifying an in vivo biological activity of said compound.

In one embodiment, the invention provides a A method of making a transgenic non-human mammal comprising: introducing into an embryo of the non-human mammal, a polynucleotide comprising a coding region for a disrupted intron of a Properdin-encoding gene; transferring the embryo into a foster mother mouse; permitting the embryo to gestate; and selecting a transgenic mouse born to said foster mother mouse, wherein said transgenic non-human mammal is characterized in that it has a decreased activation of AP-compliment when compared to a non-transgenic mammal.

### BRIEF DESCRIPTION OF THE DRAWINGS

The invention will be better understood from a reading of the following detailed description taken in conjunction with the drawings in which like reference designators are used to designate like elements, and in which:

FIG. 1 shows generation of properdin^{-/-} mice. A. Schematic representation of the mouse properdin gene locus. Vertical columns symbolize exon (E) locations. Horizontal rectangle box indicates the location of cDNA probe used for ES cell screening. B. Targeting vector. Big arrowheads represent LoxP sites and small arrowheads represent FRT sites. Neo: neomycin, DT: diphtheria toxin. C. Actual recombinant properdin gene locus. D. Expected restriction fragment lengths of wild-type and recombinant alleles and representative Southern blot screening result of ES cells after Hinc II and ScaI digestion. E. Northern blot analysis of properdin mRNA in wild-type (WT) and properdin knockout (P^{-/-}) mouse tissues. F. Immunodiffusion analysis of properdin in plasma. Antihuman properdin antibody was placed in the center well and mouse (10 μl) and human (5 μl) plasma or purified human properdin (0.5 μg) were placed in the peripheral wells. A precipitation line between the center and a peripheral well indicates the presence of properdin in the testing sample;

FIG. 2 shows rescue of properdin gene knockout by NEO deletion. A. Schematic diagram showing expected recombinant properdin gene locus after FLPe-mediated NEO deletion. B. PCR genotyping of 7 mice derived from properdin^{-/-}×FLPe-transgenic mouse crossing. Using LoxP or FLPe-specific primers, two mice (#1 and #5) were identified as having recombinant properdin gene and 4 mice (#1 to #4) were FLPe transgenic. As expected, the FLPe-negative, LoxP-positive mouse (#5) contained NEO whereas the FLPe-positive, LoxP-positive mouse (#1) did not contain NEO. C. Immunodiffusion analysis of plasma properdin showing that no properdin was present in mouse #5 (properdin−/−), whereas properdin was detected in mouse #1 (knockout rescued). Antihuman properdin antibodies were placed in the center well and plasma samples for mouse #1, #2, #5, #6 (refer to panel B) were placed in the peripheral wells;

FIG. 3 shows ELISA assays of LPS-induced AP complement activation. A. ELISA detection of LPS on LPS-coated plates. B. AP complement activation by plate-bound S. typhosa LPS in wild-type (WT) or properdin knockout (P^{-/-}) mouse serum. To reconstitute AP activity in properdin−/− mouse serum, C3−/− serum or purified human properdin (hP) was pre-mixed with properdin−/− mouse serum. Alternatively, LPS-coated plates were incubated with human properdin and washed (hP coat) before exposure to properdin−/− serum. Similar assays were performed with plate-bound LPS from S. minnesota (S) (C) or E. coli (D). E. and F. ELISA assays of human properdin interaction with plate-bound LPS. Plates were first coated with different concentrations of LPS and then incubated with a fixed concentration of purified human properdin (62.5 ng/well) (E). In panel F, plates were first coated with a fixed concentration of LPS (5 μg/ml) and then incubated with increasing concentrations of purified human properdin. After washing, the amount of plate-bound properdin was detected by anti-properdin antibodies;

FIG. 4 shows Crry^{-/-} erythrocytes- and zymosan-induced AP complement activation. A. Survival of biotin-labeled Crry^{-/-} mouse erythrocytes (1×10⁹) in wild-type (WT) or properdin^{-/-} mice. The percentage of Crry^{-/-} erythrocytes in the recipient mouse 5 min after transfusion was determined by FACS and taken as 100%. B. Representative FACS analysis of C3 deposition on zymosan after incubation with WT, properdin−/− or factor B knockout (fB^{-/-}) mouse serum in Mg⁺⁺-EGTA. C. Quantitation of C3 deposition on zymosan. Experiments were performed with two serum dilutions (1:10, 1:20) and two zymosan concentrations (0.025 mg/ml, 0.125 mg/ml). N=3 mice per group and each mouse serum was assayed in duplicates. MFI: mean fluorescence intensity. P values refer to Student t test;

FIG. 5 shows CVF-induced AP and anti-OVA/OVA-induced classical pathway complement activation. A and B. Western blot analysis of C3 activation in wild-type (A) or properdin^{-/-} (B) mouse serum. Cleavage product of the C3α-chain was detected in serum treated with CVF in Mg⁺⁺-EGTA but not in untreated serum or serum treated with CVF in EDTA. C. Densitometry of cleaved and intact C3α-chain in panels A and B. D. ELISA plate assays of anti-OVA/OVA-induced classical pathway complement activation in wild-type (WT), properdin^{-/-} and factor B knockout (fB^{-/-}) mouse serum or in properdin^{-/-} serum treated with an anti-human fB antibody;

FIG. 6 shows LOS- and LPS-induced complement activation in vivo and in vitro. A and B. ELISA assays of plasma C3 activation products in wild-type (WT) and properdin^{-/-} mice 1 hr after LOS (A) or LPS (B) treatment. LOS or LPS was given at 20 mg/kg (i.p.) and PBS was used as a vehicle control. N=3 mice per group and ELISA assays were performed in duplicate wells. A wild-type mouse plasma sample treated with CVF in vitro was used as a reference for C3 activation (100%). C and D. ELISA assay of LOS- (C) or LPS-induced (D) total complement activation in wild-type (WT), properdin−/− or factor B knockout (fB^{-/-}) mouse serum in GVB⁺⁺ buffer;

FIG. 7 shows Properdin knockout mice are resistant to arthritis development;

FIG. 8 shows monoclonal antibody clone 1.1, 2.9 and 2.11 are blocking antibodies for LPS-induced alternative pathway (AP) complement activation. Monoclonal antibody 7.11 is a non-blocking antibody (Panel A) EDTA blocks AP complement and is used as a positive control here (for complement inhibition) (Panel A) Medium cont.: used as a negative control to show the inhibition is related to mAb (Panel A) Dose-response curves of mAb clone 2.9 and 2.11 (Panel B). Calf IgG indicates IgG from cell culture medium. Method: ELISA plate is coated with LPS as an AP complement activator and the assay is performed in GVB-Mg⁺⁺-EGTA buffer;

FIG. 9 shows mAb clone 2.9 and 2.11, as well as polyclonal anti-P antibody inhibited zymodan-induced AP complement activation 20 mM EDTA was used as a positive control for inhibition of zymosan-induced AP complement activation Method: zymosan was incubated with 10% normal human serum (NHS), with or without anti-P antibodies or EDTA in GVB-Mg++-EGTA and the amount of C3 deposition on zymosan was determined by FACS;

FIG. 10 shows mAb clone 2.9 and 2.11 dose-dependently inhibited human complement-mediated lysis of rabbit erythrocytes polyclonal anti-P antibody and EDTA were used as positive controls for inhibition of rabbit erythrocyte lysis Method: rabbit erythrocytes were incubated with 7.5% normal human serum in GVB-Mg++-EGTA with or without anti-P antibodies or EDTA. The degree of lysis was determined by hemoglobin release using a spectrophotometer. Cells lysed completely by hypotonic shock was used as a control (100% lysis);

FIG. 11 shows that none of the mAbs (nor a polyclonal Ab) inhibited classical pathway (CP) complement activation (Panel A) EDTA blocks CP complement and is used as a positive control here (for complement inhibition) (Panel A) Dose-response curves of clone 2.9 and 2.11 showing lack of inhibition (Panel B) Method: ELISA plate is coated with OVA/anti-OVA immune complex and the assay is performed in GVB-Mg⁺⁺ buffer;

FIG. 12 shows mAb clone 2.9 and 2.11 had no effect on fluid phase classical pathway complement activation induced by immune complex (IC) and measured by the generation of sC5b-9 samples containing no immune complex (w/o IC) or containing IC but in the presence of EDTA were used as negative controls. Normal human serum (NHS) was incubated with OVA/anti-OVA;

FIG. 13 shows that mAb clone 2.9 and 2.11 had no effect on fluid phase classical pathway complement activation induced by immune complex (IC) and measured by the generation of C3a samples containing no immune complex (w/o IC) or containing IC but in the presence of EDTA were used as negative controls. Normal human serum (NHS) was incubated with OVA/anti-OVA;

FIG. 14 shows that mAb clone 2.9 and 2.11 had no effect on human complement-mediated lysis of antibody-sensitized sheep erythrocytes, another well-established assay for the classical pathway complement activation likewise polyclonal anti-P antibody also had no effect on human complement-mediated lysis of antibody-sensitized sheep erythrocytes. In contrast, EDTA inhibited human complement-mediated lysis of antibody-sensitized sheep erythrocytes and was used as a positive control for inhibition. Method: antibody-sensitized sheep erythrocytes were incubated with 7.5% normal human serum in GVB-Mg⁺⁺ buffer with or without anti-P antibodies or EDTA. The degree of lysis was determined by hemoglobin release using a spectrophotometer. Cells completely lysed by hypotonic shock were used as a control (100% lysis); and

FIG. 15 shows that Properdin plays a critical role in ischemia reperfusion injury. Mice were subjected to renal pedicle occlusion for 22 min, followed by 24 hr reperfusion. Blood urea nitrogen (BUN) levels were measured before (0 hr) and after (24 hr) the procedure. Compared with WT mice, DAF-CD59 double knockout mice (DKO) incurred more severe injury. This exacerbation of renal injury was dependent on C3 since DKO mice deficient in C3 (DKO-C3) were similar to WT mice in their injury. Exacerbation of renal injury was also dependent on factor B since DKO mice deficient in factor B (DKO-fB) were similar to WT mice in their injury. Exacerbation of renal injury was also dependent on properdin since DKO mice deficient in properdin (DKO-P) were similar to WT mice in their injury.

### DETAILED DESCRIPTION OF THE INVENTION

This invention relates in one embodiment to selective activation of the alternative pathway (AP) using anti-Properdin antibodies. In another embodiment, the invention is directed to methods for treating an AP complement-mediated pathology or AP mediated condition in a subject by contacting the subject with an anti-Properdin antibodies. In one embodiment, properdin knockout transgenic non-human mammals and their use are provided.

In one embodiment, properdin is structurally composed of an N-terminal domain and six thrombospondin type I repeat (TSR) domains. Under physiological conditions, it exists in plasma as cyclic polymers (dimers, trimers, tetramers), formed by head to tail associations of monomers. Human properdin is encoded on the short arm of the X chromosome and its deficiency, especially when combined with C2, MBL or IgG2 deficiency, constitutes in another embodiment, a high penetrance risk factor for lethal Neisseria infections.

In another embodiment, the methods provided herein shows activator-specific requirement of properdin in AP complement activation, and demonstrate in one embodiment, the potential of properdin as an initiator of AP complement.

The ability of the immune system to discriminate between “self” and “non-self” antigens is vital to the functioning of the immune system as a specific defense against invading microorganisms. “Non-self” antigens are those antigens on substances entering or present in the body which are detectably different or foreign from the animal's own constituents, whereas “self” antigens are those which, in the healthy animal, are not detectably different or foreign from its own constituents.

In one embodiment, provided herein is a method of treating an AP complement-mediated pathology in a subject, comprising the step of administering to said subject an alternative-pathway-specific which in another embodiment spares the classical pathway, anti-Properdin antibody, thereby inhibiting the generation of a C3bBb protein. Accordingly, the methods described herein, which in one embodiment utilize the mAb's described, do not affect the classical pathway compliment.

The classical pathway is initiated in one embodiment, by antigen-antibody complexes, while the alternative pathway is activated by specific polysaccharides, often found on bacterial, viral, and parasitic cell surfaces. The classical pathway consists of components C1-C9, while the alternative pathway consists of components C3 and several factors, such as Factor B, Factor D, and Factor H. The sequence of events comprising the classical complement pathway consists of three stages: a. recognition, b. enzymatic activation, and c. membrane attack leading to cell death. The first phase of complement activation begins with C1. C1 is made up of three distinct proteins: a recognition subunit, C1q, and the serine proteinase subcomponents, C1r and C1s, which are bound together in a calcium-dependent tetrameric complex, C1r.sub.2 s.sub.2. An intact C1 complex is necessary for physiological activation of C1 to result. Activation occurs when the intact C1 complex binds to immunoglobulin complexed with antigen. This binding activates C1s which then cleaves both the C4 and C2 proteins to generate C4a and C4b, as well as C2a and C2b. The C4b and C2a fragments combine to form the C3 convertase, which in turn cleaves C3 to form C3a and C3b. Both the classical and alternative is pathways are capable of individually inducing the production of the C3 convertase to convert C3 to C3b, the generation of which is the central event of the complement pathway. C3b binds to C3b receptors present on neutrophils, eosinophils, monocytes and macrophages, thereby activating the terminal lytic complement sequence, C5-C9.

Initiation of the classical pathway begins when antibody binds antigen. C1g binds the altered Fc region of IgG or IgM that has bound antigen. Upon binding, C1r activates C1s which initiates the activation unit by cleaving a peptide from both C4 and C2. C1s thus cleaves C4 into C4a and C4b and C2 into C2a and C2b. C2a binds to C4b forming C4b2a. C4b2a, the C3 convertase, is a proteolytic enzyme. It cleaves C3 into C3b, which may bind to the activating surface, and C3a which is released into the fluid phase (9). C3 convertase has the ability to cleave many C3 molecules. This could result in the deposition of a large number of C3b molecules on the activating surface. However, due to the labile nature of C3b, very few molecules actually bind. C4b2a3b, the C5 convertase, is formed when C3 is cleaved. C5 convertase, also an enzyme, can cleave many C5 molecules into C5a and C5b.

Accordingly, this immune response system must be maintained while bacterial and other AP-compliment activators are targeted. In one embodiment, the mAb's used in the methods described herein, do not affect the activation of the CP compliment.

Since the substrate for the alternative pathway C3 convertase is C3, C3 is therefore both a component and a product of the reaction. As the C3 convertase generates increasing amounts of C3b, an amplification loop is established. In one embodiment the classical pathway also generates C3b, whereby C3b binds factor B and engages the alternative pathway. This allows in another embodiment, more C3b to deposit on a target. In one embodiment, the binding of antibody to antigen initiates the classical pathway. If antibodies latch on to bacteria, the classical pathway generates C3b, which couples to target pathogens. In one embodiment, the antibodies used in the methods and compositions described herein do not affect the AP amplification loop of the classical pathway complement.

Accordingly and in one embodiment, provided herein is a method of treating an AP complement-mediated pathology in a subject, comprising the step of administering to said subject an alternative-pathway-specific anti-Properdin antibody or its functional fragments, thereby inhibiting the generation of a C3bBb protein.

In another embodiment, provided herein is a method of inhibiting properdin-dependent, microbial antigen-, non-biological foreign surface- or altered self tissue-triggered AP complement activation in a subject, comprising the step of administering to said subject an alternative-pathway-specific, anti-Properdin antibody or its functional fragments, thereby inhibiting the generation of a C3bBb protein.

In one embodiment, antibodies are classified into different classes based on the structure of their heavy chains. These include IgG, IgM, IgA and IgE. Antibodies having the same heavy chain structure are in one embodiment, of the same “isotype”. Antibodies of the same isotype having different antigenic determinants as a result of the inheritance of different alleles are referred to in another embodiment as “allotypes”. Antigenic determinants found primarily (but not exclusively) in the hypervariable region of the antigen binding site of the antibody are referred to in one embodiment as “idiotopes”. In another embodiment, antibodies having common or shared idiotopes are considered as members of the same idiotype.

In one embodiment, antigenic determinants on the variable regions of L chain or in another embodiment, of the H chain, which are associated with antigen-binding site of an antibody are referred to in certain embodiments as “idiotypes”. In another embodiment, antibodies raised, or which react in certain embodiments against an idiotype (idiotope) are referred to as “anti-idiotypic antibodies”.

In one embodiment, the term “antibody” includes complete antibodies (e.g., bivalent IgG, pentavalent IgM) or fragments of antibodies which contain an antigen binding site in other embodiments. Such fragments include in one embodiment Fab, F(ab′)₂, Fv and single chain Fv (scFv) fragments. In one embodiment, such fragments may or may not include antibody constant domains. In another embodiment, Fab′ s lack constant domains which are required for Complement fixation. ScFvs are composed of an antibody variable light chain (V_{L}) linked to a variable heavy chain (V_{H}) by a flexible hinge. ScFvs are able to bind antigen and can be rapidly produced in bacteria or other systems. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

Antibodies of the invention can be monoclonal antibodies (mAb) in one embodiment, or polyclonal antibodies in another embodiment. Antibodies of the invention which are useful for the compositions, methods and kits of the invention can be from any source, and in addition may be chimeric. In one embodiment, sources of antibodies can be from a mouse, or a rat, a plant, or a human in other embodiments. Antibodies of the invention which are useful for the compositions, and methods of the invention have reduced antigenicity in humans (to reduce or eliminate the risk of formation of anti-human antibodies), and in another embodiment, are not antigenic in humans. Chimeric antibodies for use the invention contain in one embodiment, human amino acid sequences and include humanized antibodies which are non-human antibodies substituted with sequences of human origin to reduce or eliminate immunogenicity, but which retain the antigen binding characteristics of the non-human antibody.

In one embodiment, heavy and light chains are randomly paired during PCR construction using phage display technique. In one embodiment, the term “phage display” or “phage display technique” refers to a methodology that utilizes fusions of nucleic acid sequences encoding foreign polypeptides of interest to sequences encoding phage coat proteins, in order to display the foreign polypeptides on the surface of bacteriophage particles. In another embodiment, applications of the technology include the use of affinity interactions to select particular clones from a library of polypeptides (such as the anti properdin monoclonal antibodies provided in the compositions described herein), the members of which are displayed on the surfaces of individual phage particles. Display of the polypeptides is due in one embodiment, to expression of sequences encoding them from phage vectors into which the sequences have been inserted. In one embodiment, a library of polypeptide encoding sequences are transferred to individual display phage vectors to form a phage library that can be used in another embodiment, to screen for polypeptides of interest.

In one embodiment, the term “phage surface protein” refers to any protein normally found at the surface of a bacteriophage that can be adapted to be expressed as a fusion protein with a heterologous polypeptide and still be assembled into a phage particle such that the polypeptide is displayed on the surface of the phage.

As will be understood by those skilled in the art, the immunologically binding reagents encompassed by the term “antibodies or their fragment” extend in certain embodiments, to all antibodies from all species including dimeric, trimeric and multimeric antibodies; bispecific antibodies; chimeric antibodies; human and humanized antibodies; recombinant and engineered antibodies, and fragments thereof. The term “antibodies or their fragment” refers in another embodiment to any antibody-like molecule that has an antigen binding region, and this term includes small molecule agent fragments such as Fab′, Fab, F(ab′)₂, single domain antibodies (DABs), Fv, scFv (single chain Fv), linear antibodies, diabodies, and the like. The techniques for preparing and using various antibody-based constructs and fragments are well known in the art In one embodiment, the anti-properdin fragment used in the methods and compositions described herein, is Fc, or Fab, F(ab′), F(ab′)₂ or a combination thereof in other embodiments. In another embodiment, the anti-properdin fragment used in the methods and compositions described herein, is Fc, or Fab, F(ab′), F(ab′)₂ or a combination thereof in other embodiments.

The term “antibody fragment” also includes any synthetic or genetically engineered protein that acts like an small molecule agent by binding to a specific antigen to form a complex. In one embodiment, antibody fragments include isolated fragments, “Fv” fragments, consisting of the variable regions of the heavy and light chains, recombinant single chain polypeptide molecules in which light and heavy chain variable regions are connected by a peptide linker (“sFv proteins”), and minimal recognition units consisting of the amino acid residues that mimic the hypervariable region. In one embodiment, the antibody is a variable regions of the heavy and light chains, or recombinant single chain polypeptide molecules in which light and heavy chain variable regions are connected by a peptide linker (“sFv proteins”), and minimal recognition units consisting of the amino acid residues that mimic the hypervariable region in other embodiments.

In one embodiment, the anti-properdin mAbs used in the methods and compositions described herein, selectively inhibit AP complement activation and have no effect on the AP amplification loop of the CP. In another embodiment, the mAbs described herein are distinct from the anti-properdin mAbs developed and which inhibit both AP and CP complement.

Accordingly, in one embodiment, provided herein is a method of treating an AP complement-mediated pathology in a subject, or properdin-dependent, microbial antigen-, non-biological foreign surface- or altered self tissue-triggered AP complement activation, comprising the step of administering to said subject an alternative-pathway-specific anti-Properdin antibody, thereby inhibiting the generation of a C3bBb protein, whereby the antibody does not affect the AP amplification loop of the classical pathway complement.

In one embodiment, properdin is indispensable for LPS- and LOS-induced AP complement activation and in another embodiment, for AP complement-mediated extravascular hemolysis of Crry-deficient erythrocytes. In one embodiment, zymosan-induced AP complement activation is moderately impaired by properdin deficiency. In another embodiment, properdin plays a negligible, or in another embodiment, does not have any role in CVF- and classical pathway-triggered AP complement amplification. In one embodiment, properdin is more relevant to independent AP complement initiation than to AP complement amplification secondary to other activation pathways. In another embodiment, the need for properdin in AP complement initiation is variable and depends on the nature of the activating surface. In one embodiment both foreign and endogenous AP complement activators critically depend on properdin for their activity.

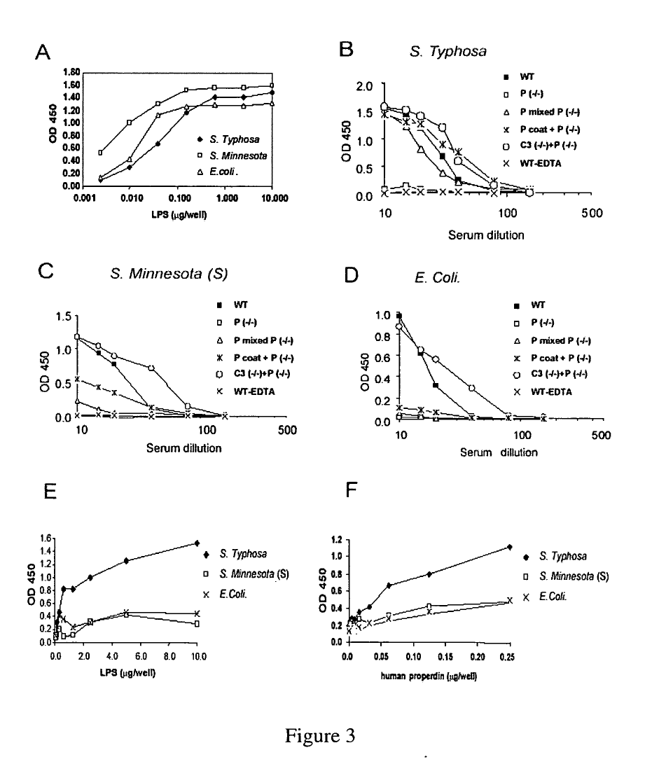

In one embodiment, AP activation on a given surface represents the balance between properdin-dependent promotion via C3bBb stabilization and factor H (fH)-dependent inhibition of C3 ‘tick-over’. In another embodiment, an AP activator for which properdin is not essential may have limited interaction with fH and, as a result of lacking sufficient fH-dependent inhibition, spontaneous C3 activation and amplification could occur as a default process without the help of properdin. In another embodiment purified human properdin, restores S. typhosa LPS-induced, but not S. minnesota (S) or E. coli LPS-induced, AP complement activity in properdin^{-/-} serum (FIG. 3).

In another embodiment, properdin binds to an AP activator directly, or in another embodiment, via initially deposited C3b, directing complement activation by serving as a platform for new C3bBb assembly. In one embodiment, surface-bound properdin promotes C3bBb formation; and in another embodiment the ability of human properdin to restore LPS-induced AP complement activity in properdin^{-/-} mouse serum correlates with its LPS-binding affinity (FIG. 3). In one embodiment LPS-bound human properdin activates AP complement in the serum of properdin-deficient subjects in the absence of any solution properdin (FIG. 3).

In one embodiment, by virtue of its binding affinity towards an activating surface, properdin acts as an obligatory pattern recognition molecule for AP complement initiation. In another embodiment, zymosan causes vigorous AP complement activation in serum of properdin is deficient subject, indicating that other factor(s) act in a similar activator-specific manner for AP complement initiation.

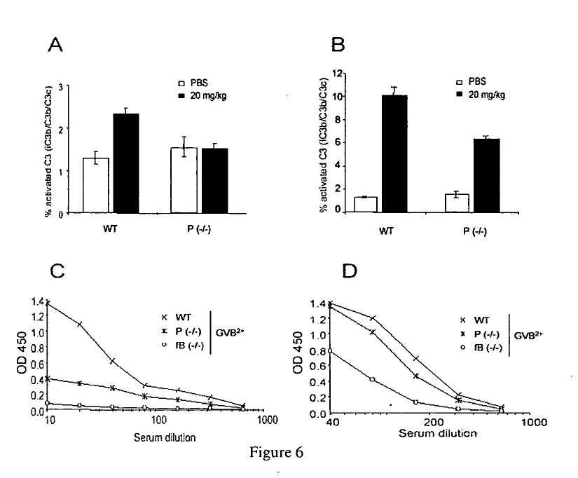

In one embodiment, properdin-deficient individuals are susceptible to bacterial infection. In another embodiment, LOS-induced complement activation in vivo is abolished in properdin-deficient individuals whereas that induced by LPS was only partially impaired (FIG. 6). In another embodiment, AP is the predominant pathway in LOS- but not LPS induced complement activation. In one embodiment, properdin deficiency, especially when combined in another embodiment, with low antibody or in another embodiment, with mannose-binding lectin levels, abrogates complement-mediated bactericidal activity towards LOS-containing meningitides.

In one embodiment, properdin plays a role in host defense. In another embodiment, properdin produced by leukocytes at sites of inflammation initiates AP complement and amplifies tissue injury.

Accordingly and in one embodiment, provided herein is a method of treating an AP complement-mediated pathology in a subject, comprising the step of administering to said subject an inhibitor of an activity of a Properdin protein, thereby treating an AP complement-mediated pathology in a subject.

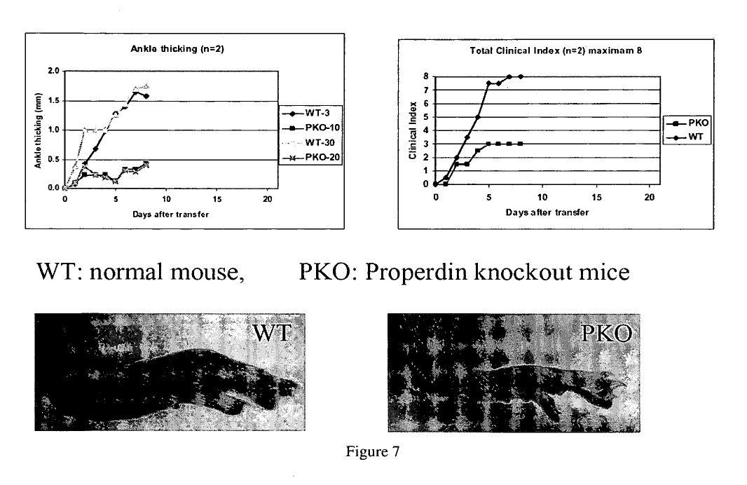

In one embodiment, the AP complement-mediated pathology treated by contacting the subject with an inhibitor of an activity of a Properdin protein, is age-related macular degeneration (AMD). In another embodiment, the AP complement-mediated pathology is ischemia reperfusion injury. In another embodiment, the AP complement-mediated pathology is arthritis (see FIG. 7). In another embodiment, the AP complement-mediated pathology is paroxysmal nocturnal hemoglobinuria (PNH) syndrome. In another embodiment, the AP complement-mediated pathology is atypical hemolytic uremic (aHUS) syndrome.

In one embodiment, the activity of a Properdin protein inhibited using the A method of treating an AP complement-mediated pathology in a subject, or in another embodiment, AP complement activation induced by a lipooligosaccharide (LOS); or in another embodiment, inhibiting a pattern recognition receptor-mediated AP complement activation; or in another embodiment inhibiting an initiation of an alternate pathway (AP) complement activation, is a generation of a C3bBb protein. In another embodiment, the inhibitor of properdin activity used in the methods provided herein, does not inhibit a classical pathway-triggered complement activation in said subject and in one embodiment, does not inhibit a lectin pathway-triggered, zymosan-induced, or cobra venom factor-induced AP complement activation. In one embodiment, the inhibitor of properdin activity used in the methods provided herein, does not inhibit a lectin pathway-triggered, zymosan-induced, or cobra venom factor-induced AP complement activation.

In one embodiment, the term “complement activation” refers to complement amplification. In another embodiment, the inhibitor of an activity of a Properdin protein used in the methods provided herein, impedes activation of the AP complement. In one embodiment, inhibitor as used in the method of treating or inhibiting or suppressing or reducing symptoms of pathologies that are AP complement-mediated, comprising the step of administering to said subject an inhibitor of an activity of a Properdin protein, may be an antibody, such as, in another embodiment, an antibody that binds the Properdin protein, or small molecule, peptide, peptidomimetic, cyclical peptide and their combination in other embodiments.

In one embodiment, provided herein are methods of treating pathologies that are AP complement-mediated, comprising the step of administering to said subject a composition that reduces a Properdin protein level in a tissue or body fluid of said subject. In another embodiment, provided herein are methods of inhibiting an alternate pathway (AP) complement-mediated destruction of red blood cells or platelet in a subject, comprising the step of administering to said subject the inhibitor of an activity of a Properdin protein described herein.

In another embodiment, a method of present invention exhibits the advantage that it preserves ability of the subject to combat an infection using the classical complement activation pathway. In another embodiment, provided herein is a method of inhibiting an AP complement activation induced by bacterial lipooligosachamide (LOS) in a subject, comprising the step of administering to said subject an inhibitor of an activity of a Properdin protein, thereby inhibiting an AP complement activation induced by bacterial LOS in a subject. In another embodiment, the inhibitor used in the methods of inhibiting an AP complement activation induced by bacterial lipooligosachamide (LOS) in a subject, is any of the inhibitor embodiments described herein. Accordingly and in another embodiment, provided herein is a method of inhibiting an AP complement activation induced by a bacterial LPS. In one embodiment, the AP complement activation is induced by S. typhosa LPS, and the inhibitors used in the methods provided herein do not inhibit AP complement activity induced by S. minnesota (S) or E. coli LPS, or both.

In one embodiment, provided herein is a method of inhibiting a pattern recognition receptor-mediated AP complement activation in a subject, comprising the step of administering to said subject an inhibitor of an activity of a Properdin protein, thereby inhibiting a pattern recognition receptor-mediated AP complement activation in a subject.

In another embodiment, the AP complement activation results from recognition by said pattern recognition receptor; of a microbial antigen that is muramyl di-peptide (MDP). In another embodiment, the AP complement activation results from recognition by said pattern recognition receptor; of a microbial antigen is a CpG motif from bacterial DNA. In another embodiment, the AP complement activation results from recognition by said pattern recognition receptor; of a microbial antigen is peptidoglycan. In another embodiment, the AP complement activation results from recognition by said pattern recognition receptor; of a microbial antigen is lipoteichoic acid. In another embodiment, the AP complement activation results from recognition by said pattern recognition receptor; of a microbial antigen is an outer surface protein A from Borrelia burgdorferi. In another embodiment, the AP complement activation results from recognition by said pattern recognition receptor; of a microbial antigen is a synthetic mycoplasmal macrophage-activating lipoprotein-2, tripalmitoyl-cysteinyl-seryl-(lysyl)-3-lysine (P3CSK4). In another embodiment, the AP complement activation results from recognition by said pattern recognition receptor; of a microbial antigen is dipalmitoyl-CSK4 (P2-CSK4). In another embodiment, the AP complement activation results from recognition by said pattern recognition receptor; of a microbial antigen is monopalmitoyl-CSK4 (PCSK4). In another embodiment, the AP complement activation results from recognition by said pattern recognition receptor; of a microbial antigen is amphotericin B. In another embodiment, the AP complement activation results from recognition by said pattern recognition receptor; of a microbial antigen is a triacylated or diacylated bacterial polypeptide. In another embodiment, the AP complement activation results from recognition by said pattern recognition receptor; of a microbial antigen is a combination thereof.

In one embodiment, provided herein is a method of inhibiting an initiation of an alternate pathway (AP) complement activation in a subject, comprising the step of administering to said subject an inhibitor of an activity of a Properdin protein, thereby inhibiting an initiation of an AP complement activation in a subject.

In another embodiment, a method of present invention exhibits the advantage that it preserves ability of the subject to activate complement via the classical activation pathway. In another embodiment, a method of present invention exhibits the advantage that it preserves ability of the subject to activate complement via the lectin activation pathway.

In one embodiment, provided herein is a transgenic knock-out animal whose genome comprises a homozygous disruption in an endogenous properdin gene, wherein said homozygous disruption prevents function of properdin and results in said transgenic knockout mouse exhibiting decreased AP-compliment as compared to a wild-type mouse.

In another embodiment, provided herein is a method for selecting a potential therapeutic compound for use in treating an AP complement-mediated pathology in a subject, or in another embodiment, AP complement activation induced by a lipooligosaccharide (LOS); or in another embodiment, inhibiting a pattern recognition receptor-mediated AP complement activation; or in another embodiment inhibiting an initiation of an alternate pathway (AP) complement activation, comprising: a) administering the compound to a wild-type animal or an animal having an AP complement-mediated pathology, or in another embodiment, AP complement activation induced by a lipooligosaccharide (LOS); or in another embodiment, a pattern recognition receptor-mediated AP complement activation; or in another embodiment an initiation of an alternate pathway (AP) complement activation; b) measuring the resulting phenotype of wild-type animal or the animal having the an AP complement-mediated pathology, or in another embodiment, AP complement activation induced by a lipooligosaccharide (LOS); or in another embodiment, a pattern recognition receptor-mediated AP complement activation; or in another embodiment an initiation of an alternate pathway (AP) complement activation; and c) comparing the resulting phenotype of the wild-type animal or the animal having an AP complement-mediated pathology, or in another embodiment, AP complement activation induced by a lipooligosaccharide (LOS); or in another embodiment, a pattern recognition receptor-mediated AP complement activation; or in another embodiment an initiation of an alternate pathway (AP) complement activation; to the phenotype of a properdin^{-/-} knockout animal.

In one embodiment, provided herein is a method of making a transgenic non-human mammal comprising: introducing into an embryo of the non-human mammal, a polynucleotide comprising a coding region for a disrupted intron of a Properdin-encoding gene; transferring the embryo into a foster mother mouse; permitting the embryo to gestate; and selecting a transgenic mouse born to said foster mother mouse, wherein said transgenic non-human mammal is characterized in that it has a decreased activation of AP-compliment when compared to a non-transgenic mammal.

In another embodiment, provided herein is a transgenic non-human mammal and progeny thereof whose genome comprises a disruption of a Properdin-encoding gene such that the mammal lacks or has reduced levels of functional Properdin.

In one embodiment, provided herein is a transgenic non-human mammal and progeny thereof whose genome comprises a disruption of a Properdin-encoding gene such that the mammal lacks or has reduced levels of functional Properdin, wherein a neomycine cassette (NEO) is inserted between exons 5 and 6 of said Properdin gene, resulting in one embodiment, in the disruption of an intron between exons 5 and 6.

In another embodiment, provided herein is a cell, organ, tissue or their combination, obtained from the transgenic non-human mammal described herein. In one embodiment, provided herein is a method of culturing the transgenic cells derived from the transgenic non-human mammals described herein, comprising the steps of: providing the cell of the non-human transgenic mammal and culturing said cell under conditions that allow growth of said cell.

In one embodiment, provided herein is a method of making a transgenic non-human mammal comprising: introducing into an embryo of the non-human mammal, a polynucleotide comprising a coding region for a disrupted intron of a Properdin-encoding gene; transferring the embryo into a foster mother mouse; permitting the embryo to gestate; and selecting a transgenic mouse born to said foster mother mouse, wherein said transgenic non-human mammal is characterized in that it has a decreased activation of AP-compliment when compared to a non-transgenic mammal. In one embodiment step of selecting a transgenic mouse born to said foster mother mouse in the methods described herein, comprises mating two selected transgenic mice; permitting the embryos to gestate; and selecting a transgenic mouse born to a transgenic mother. In one embodiment, the method of making a transgenic non-human mammal is repeated for more than one generation.

In another embodiment, the inhibitor used in the methods provided herein is identified by the method for selecting a potential therapeutic compound using the transgenic animal described herein.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

### Properdin Gene Targeting

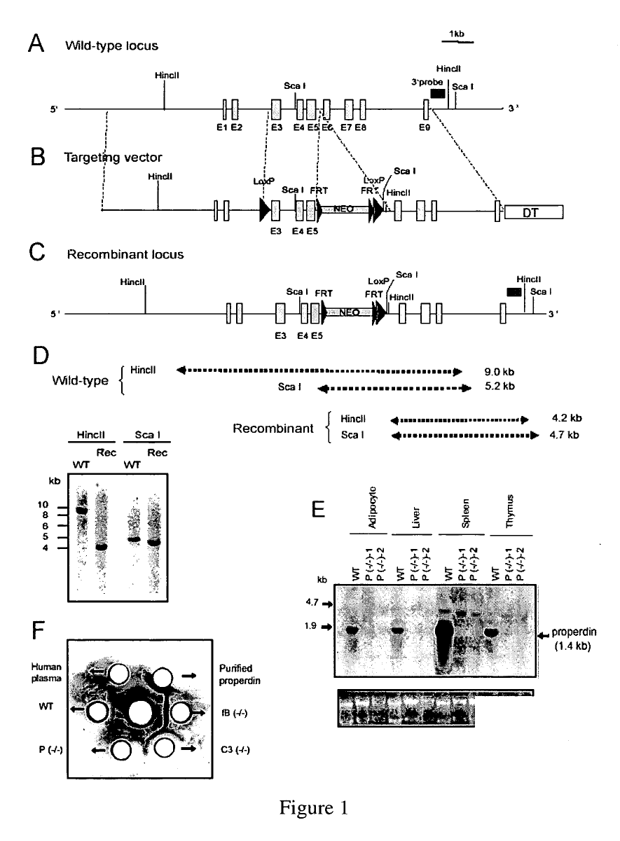

To construct the targeting vector, pNDI vector was used, which contains neomycin (NEO) and diphtheria toxin (DT) as a positive and negative selection marker, respectively (kindly provided by Dr Glen Radice, University of Pennsylvania). This vector contains two LoxP sites for Cre recombinase-mediated gene excision, and the NEO was flanked by two FRT sites for potential excision by the FLPe recombinase. Properdin gene fragments were amplified by PCR using 129/Sv mouse genomic DNA as template and with The Expand Long Template PCR System (Roche). For the 3′ homologous arm, a 3.5 kb gene fragment containing exon 6-9 was amplified using 5′-CTCGAGCATTCATCTTTGCCAGAAATC-3′ (SEQ ID NO. 1) and 5′-TCCCCATACTCAGCACTATTG-3′ (SEQ ID NO. 2) as primers, cloned into the PCR 2.1 vector (Invitrogen, CA), and then subcloned into the EcoRI site in pND1 (downstream of the NEO cassette, FIG. 1B). For the 5′ homologous arm, two fragments, a 4 kb NotI-EcoRV fragment containing exon 1-2 and a 1.6 kb EcoR V-XhoI fragment containing exon 3-5, as well as incorporating a 34 by LoxP site (5′-ATAACTTCGTATAATGTATGCTATACGAAGTTAT-3′ (SEQ ID NO. 3)), were amplified using the following primer pairs: 5′-GATATCATAACTTCGTATAATGT-ATGCTATACGAAGTTATGTTCAATCACCCACCATCCCT-3′ (SEQ ID NO. 4) and 5′-CTCGAGCATTCATCTTTGCCAGAAATC-3′ (SEQ ID NO. 5); 5′-GCGGCCGCATTCC-GGCTGTATCTGAGTC-3′ (SEQ ID NO. 6) and 5′-GATATCAGGAAGAAGTGAA-TATACAGG-3′ (SEQ ID NO. 7). These 2 pieces were cloned into the pND1 vector at NotI-XhoI sites (upstream of the Neo) in a 3-piece ligation experiment.

The targeting vector was linearized by Not I digestion before transfection. ES cells were selected with G418 (0.2 mg/ml) and positive clones were screened by Southern blot using Hinc II- and Sca I-digested genomic DNAs and a 513 by probe located 3′ to the right homologous arm (FIG. 1, A-D). ES cell culture, vector transfection, clone selection and chimera mouse production were carried out as described. Male littermates were used in all experiments. For PCR genotyping, 5′-GGGTGGGATTAGATAAATGCC-3′ (P1, NEO-specific; (SEQ ID NO. 8)) and 5′-CAAGGTACGGCTTTGTTACACA-3′ (P2, properdin-specific; (SEQ ID NO. 9)) were used for NEO detection (700 by product), 5′-ATAACTTCGTATAATGTATGCTATACGAAGTTAT-3′ (SEQ ID NO. 10) and P2 were used for LoxP detection (400 by product). 5′-CACTGATATTGTAAGTAGTTTGC-3′ (SEQ ID NO. 11) and 5′-CTAGTGCGAAGTAGTGATCAGG-3′ (SEQ ID NO. 12) were used for FLPe transgene detection. All animal experiments were approved by the Institutional Animal Care and Use Committee of the University of Pennsylvania.

Other Mice and Reagents.

C3^{-/-} and FLPe-Tg (B6; SJL-Tg(ACTFLPe)9205 Dym/J) mice were from the Jackson Laboratory (Bar Harbor, Me.). fB^{-/-} mice, rabbit anti-OVA and rabbit anti-C3c antibodies were kindly provided by Dr J. Lambris (University of Pennsylvania). Crry^{-/-}C3^{-/-} mice were kindly provided by Dr H. Molina (Washington University). Zymosan A (Saccharomyces cerevisiae), S. Typhosa, S. Minnesota (S), E. Coli 026:B6 LPS, OVA and HRP anti-mouse IgG were from Sigma-Aldrich. Human properdin was from Quidel (San Diego, Calif.). Anti-core LPS mAb, WN1 222-5, was from Cell sciences (Canton, Mass.). Goat anti-human properdin and fB antibodies were from Complement Technologies (San Diego, Calif.). HRP goat anti-C3 antibody was from MP Biomedicals (Solon, Ohio). N. meningitidis LOS was kindly provided by Dr. Sanjay Ram (University of Massachusetts, Worcester).

Northern Blot and Immunodiffusion Assay.

Northern blot analysis was performed as described [Miwa T, Zhou L, Hilliard B, Molina H, Song W C. Crry, but not CD59 and DAF, is indispensable for murine erythrocyte protection in vivo from spontaneous complement attack. Blood. 2002; 99:3707-3716.] using a 700 by mouse properdin cDNA as a probe. Plasma properdin was detected by immunodiffusion assays as described in Current Protocols in Immunology (John Wiley and Sons, Inc.) using anti-human properdin antibodies.

Detection of Plate-Bound LPS.

To compare the plate-coating efficiency of LPS from different bacterial species (S. typhosa, S. minnesota and E. coli.), plates were coated with diluted concentrations of LPS. After blocking with BSA (10 mg/ml) for 1 h, plates were washed with PBS and incubated for 1 h with WN1 222-5 (0.5 μg/ml), a murine anticore LPS mAb, followed by detection with HRP-anti mouse IgG (1:6000). BSA-coated wells were used as background controls.

ELISA Assays of Complement Activation.

Plates were coated with LPS (2 μg/well) or OVA/anti-OVA immune complex for complement activation assays as described previously [Sfyroera G, Katragadda M, Morikis D, Isaacs S N, Lambris J D. Electrostatic modeling predicts the activities of orthopoxvirus complement control proteins. J Immunol. 2005; 174:2143-2151.]. Diluted mouse serum (50 μl per well) was incubated on plates at 37° C. for 1 h followed by detection of plate-bound activated C3 using HRP anti-mouse C3 antibody (1:4000). AP activity was assayed in Mg⁺⁺-EGTA and total or classical pathway activity was assayed in GVB⁺⁺. For reconstitution experiments, 1:10 diluted (in Mg⁺⁺-EGTA) C3^{-/-} serum was pre-mixed (at 1:1 ratio) with variously diluted properdin−/− serum. Alternatively, human properdin was added to properdin−/− serum (62.5 ng to 50 μl serum) or used to pre-treat LPS-coated plates (62.5 ng in 25 μl Mg⁺⁺-EGTA, 1 hr at 37° C., followed by washing). To deplete fB, properdin^{-/-} serum was pre-incubated with antihuman fB IgG (8 μg per 1 μl serum), followed by centrifugation to remove anti-fB/fB immune complexes.

LPS-Properdin Binding.

In the first assay, plates were coated with different concentrations of LPS, blocked with BSA (10 mg/ml) and then incubated with purified human properdin (2.5 μg/ml in Mg⁺⁺-EGTA, 25 μl/well) at RT for 1 hr, followed by washing and detection with biotinylated anti-properdin IgG (2 μg/ml) and avidin-HRP (1:10,000). LPS-coated wells not treated with properdin were used as background controls. In the second assay, plates were coated with a fixed concentration of LPS (5 μg/well) and then incubated with increasing concentrations of purified human properdin. Measurement of AP activation on zymosan. Zymosan (0.025 or 0.125 mg/ml) was incubated with serum in Mg⁺⁺-EGTA for 15 mins at 370 C. 22 and C3 deposition was assessed by FACS as described [Kim D D, Miwa T, Song W C. Retrovirus-mediated over-expression of decayaccelerating factor rescues Crry-deficient erythrocytes from acute alternative pathway complement attack. J Immunol. 2006; 177:5558-5566.].

CVF Treatment In Vitro.

Serum (5 μl) was incubated with 0.01 μg or 0.3 μg CVF for various lengths of time. After incubation, 0.5 μl serum was run on an 8% gel under reducing conditions and subjected to Western blot analysis as described [Xu Y, Ma M, Ippolito G C, Schroeder H W, Jr., Carroll M C, Volanakis J E. Complement activation in factor D-deficient mice. Proc Natl Acad Sci USA. 2001; 98:14577-14582.] using HRP-conjugated rabbit anti-mouse C3 antibody. C3 cleavage was quantified by densitometry scanning of activated and intact C3α-chain.

Erythrocyte Transfusion and Survival Assay.

Sensitivity of Crry-deficient mouse erythrocytes to AP complement attack in vivo was assayed as described [Miwa T, Zhou L, Hilliard B, Molina H, Song W C. Crry, but not CD59 and DAF, is indispensable for murine erythrocyte protection in vivo from spontaneous complement attack. Blood. 2002; 99:3707-3716.].

In Vivo Complement Activation Induced by LOS or LPS.

Mice were injected (i.p.) with 20 mg/kg N. meningitidis LOS or S. typhosa LPS. Plasma levels of activated C3 were determined at 1 hr after treatment as described [Mastellos D, Prechl J, Laszlo G, et al. Novel monoclonal antibodies against mouse C3 interfering with complement activation: description of fine specificity and applications to various immunoassays. Mol Immunol. 2004; 40:1213-1221.].

### Example 1

### Generation of a Properdin Knockout (Properdin^{-/-}) Mouse

The mouse properdin gene is located on the X chromosome and is composed of 9 exons (http://www.informatics.jax.org/searches/accession_report.cgi?id=MGI:97545)(FIG. 1A). The original plan was to generate a conditional properdin gene knockout mouse so that the Jo significance of its tissue-specific production could be studied. To achieve this goal, a targeting vector was constructed by cloning 5′ and 3′ homologous arm sequences into the pND1 vector as illustrated in FIG. 1B. According to this strategy, after correct targeting the neomycin cassette (NEO) would be inserted between exon 5 and 6 of the properdin gene, and exon 3-5 would be flanked by two LoxP sites (FIG. 1B), allowing them to be deleted by tissue-specific Cre recombinase. Exon 3-5 was targeted for deletion because mutations in exon 4-6 of the human properdin gene are associated with properdin deficiency. Targeted embryonic stem (ES) cells were selected by Southern blot analysis after Hinc II and Sca I digestion of genomic DNA (FIGS. 1, C and D), using a 513 by probe located outside the 3′ homologous arm. 7 positive ES cell clones were obtained and two of them were used for chimeric mice production. Chimeras derived from both ES cell clones successfully transmitted the mutation through the germline.

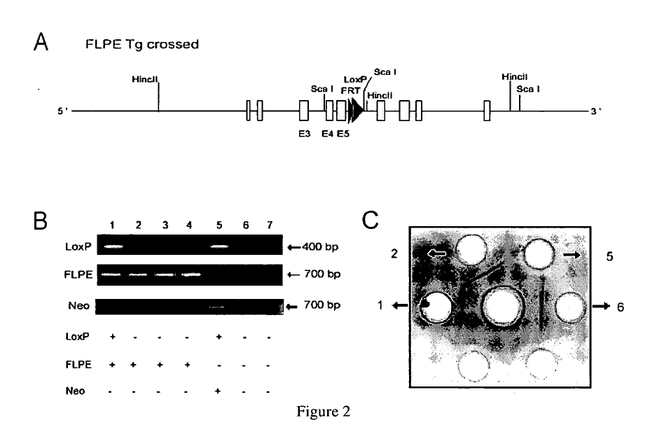

Subsequent analysis of the recombinant properdin gene allele, both in the mutant mice and in the two ES cell clones used to generate them, confirmed NEO insertion at the intended location but failed to detect the 5′ LoxP sequence (FIG. 1C). The latter outcome was unexpected but most likely occurred as a result of homologous recombination in the sequence downstream (i.e. exon 3-5) rather than upstream (i.e. exon 1-2 and 5′ flanking region) of the 5′LoxP site (FIG. 1A, B). Nevertheless, Northern blot and real-time PCR analysis detected no properdin mRNA expression in various tissues of the mutant mice (FIG. 1E), and immunodiffusion analysis confirmed the lack of properdin protein in their plasma (FIG. 1F). These results indicated that NEO insertion into the small intron (201 bps) between exon 5 and 6 might have unintentionally disrupted the properdin gene. To verify this conclusion, the properdin mutant mouse was crossed with the FLPe transgenic mouse. The NEO cassette in the targeting construct was flanked by two FRT sites which could be recognized by the FLPe recombinase. Expression of the FLPe recombinase eliminated NEO from the genome of properdin gene-targeted mice with corresponding recovery of properdin protein in their plasma (FIG. 2). Thus, by NEO insertion into the 5^{th} intron, a global properdin gene knockout mouse (properdin^{-/-}) was unexpetedly created.

### Example 2

### Abrogation of LPS-Induced AP Complement Activation in Properdin^{-/-} Mouse Serum

To assess AP complement activity in properdin^{-/-} mouse serum, an ELISA assay was used to measure LPS-induced complement activation in Mg⁺⁺-EGTA. LPS was coated onto 96-well plates and after exposure to mouse serum, the level of C3 deposition on the plates was determined. Using a broadly cross-reacting anti-core LPS mAb, it was first confirmed that LPS from three different bacteria species, S. typhosa, S. minnesota (S) and E. coli bound to ELISA plate with similar avidity (FIG. 3A). FIG. 3B-D shows that these LPS all activated AP complement in wild-type (WT) mouse serum. In contrast, the same LPS did not cause appreciable AP complement activation in properdin^{-/-} mouse serum or in WT mouse serum treated with EDTA (negative control) (FIG. 3B-D). Addition of C3^{-/-} mouse serum (as a source of murine properdin) or purified human properdin to properdin^{-/-} mouse serum restored S. typhosa LPS-induced AP complement activity to WT or higher levels (FIG. 3B). Importantly, pre-treatment of S. typhosa LPS coated plates with human properdin followed by washing also reconstituted AP complement activation in properdin^{-/-} mouse serum (FIG. 3B). This result suggested that purified human properdin was able to bind to S. typhosa LPS with sufficient affinity and that immobilized LPS-bound properdin activated AP complement in the absence of solution properdin.

By pre-mixing with C3⁺⁺ mouse serum, similar reconstitution of S. minnesota (S) and E. coli LPS-induced AP complement activity were observed in properdin^{-/-} serum (FIG. 3C, D). Surprisingly, unlike the observation with S. typhosa LPS, purified human properdin only partially restored S. minnesota (S) and E. coli LPS-induced AP complement activity in properdin^{-/-} serum, irrespective of whether the protein was added to properdin−/− serum or used to pre-treat LPS-coated plates (FIG. 3C, D). Next, the relative binding affinity of purified human properdin was compared to LPS of the three bacteria species. FIG. 3E, F shows that human properdin did not bind to the plate in the absence of LPS coating, but it displayed a clear LPS concentration- and properdin concentration-dependent binding to S. typhosa LPS. This contrasted starkly with its weak binding to S. minnesota (S) and E. coli LPS. Thus, the ability of human properdin to restore LPS-dependent AP complement activity in properdin^{-/-} serum correlated with its binding affinity to LPS.

### Example 3

### AP Activation on Non-Protected Autologous Cells Also Depends on Properdin

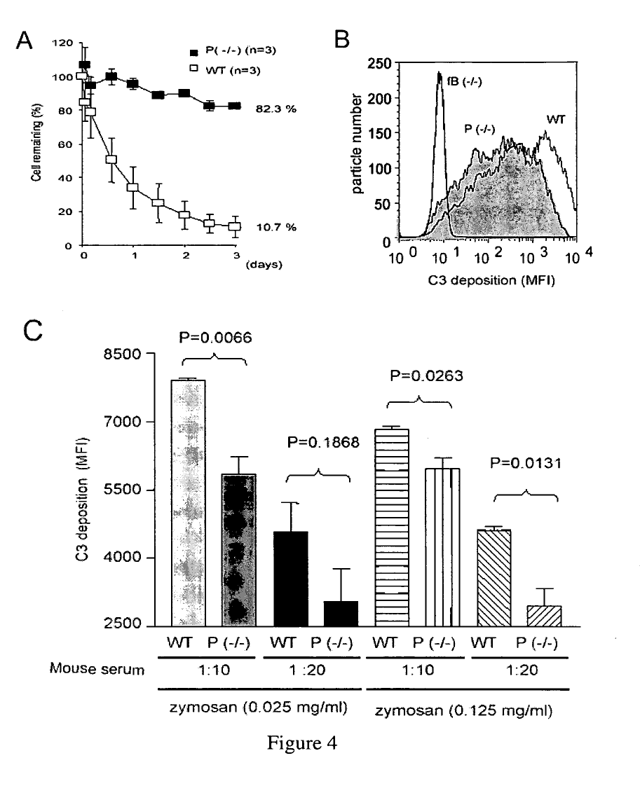

To assess the role of properdin in this process, C^{rry}-deficient mouse erythrocytes were transfused into WT and properdin^{-/-} mice. FIG. 4A shows that Crry-deficient erythrocytes were rapidly eliminated in WT but not properdin−/− recipients. Thus, spontaneous AP complement activation on non-protected autologous cells also required properdin to be initiated.

### Example 4

### Properdin is not Essential for Zymosan- or CVF-Induced AP Complement Activation

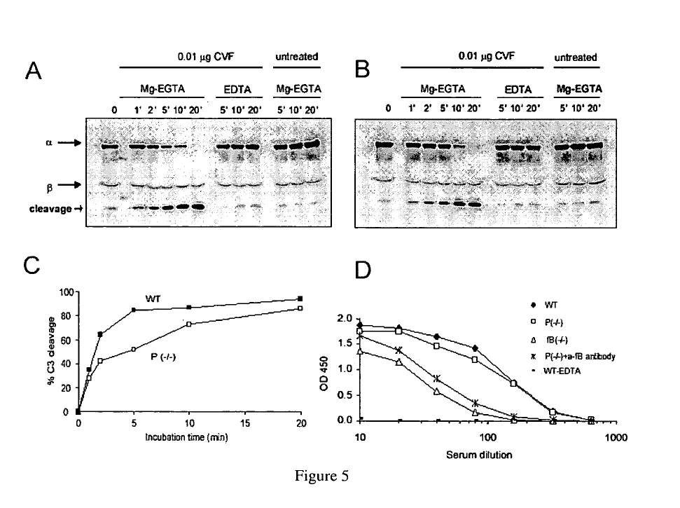

Zymosan was incubated with WT or properdin^{-/-} mouse serum and assessed AP complement activation by FACS analysis of C3 deposition. As shown in FIG. 4B,C, it was found that zymosan-induced AP complement activation was only partially impaired in properdin^{-/-} serum. This was in clear contrast with factor B-deficient (fB^{-/-}) mouse serum which supported no AP complement activation (FIG. 4B). Cobra-venom factor (CVF) binds factor B with high affinity and CVFBb acts as a stable C3 convertase to cause extensive AP complement activation in vivo and in vitro. To evaluate the role of properdin in CVF-induced AP complement activation, WT or properdin^{-/-} mouse serum was treated with CVF and analyzed C3 activation kinetics by Western blot analysis. It was found that CVF (0.01 μg for 5 μl serum) induced complete C3 cleavage within 20 min in both types of sera, but the C3 activation kinetics in the properdin^{-/-} serum appeared to be slightly delayed (FIG. 5A-C). However, no difference were observed between WT and properdin^{-/-} sera when a higher dose of CVF (0.3 μg for 5 μl serum) was used. In this case, complete C3 cleavage was achieved within 1 min of CVF treatment in both sera. Thus, properdin plays an insignificant role in CVF-induced AP complement activation.

### Example 5

### Properdin Plays a Negligible Role in Classical Pathway-Triggered AP Complement Amplification

Activation of the classical and lectin pathways inevitably initiates the AP pathway. To determine if properdin plays a role in classical pathwaytriggered AP complement amplification, a plate-based assay was used to measure anti-OVA/OVA-induced complement activity in WT and properdin^{-/-} sera. fB^{-/-} serum was fused as a negative control for AP amplification in this experiment. As shown in FIG. 5D, a significant difference was observed in complement activation between WT and fB^{-/-} mouse serum, confirming that AP amplification contributes substantially to the overall complement activation initiated via the classical pathway. In contrast, anti-OVA/OVA-induced complement activation was minimally reduced in properdin^{-/-} serum (FIG. 5D). This result suggested that either the AP amplification loop was largely intact in properdin^{-/-} mice or there was compensatory up-regulation in the activity of the classical pathway C3 convertase. To distinguish these two possibilities, fB was depleted from properdin^{-/-} serum using anti-human fB antibodies. It was found that depletion of fB from properdin^{-/-} serum reduced anti-OVA/OVA-induced complement activation to a level that was comparable to that observed in fB^{-/-} serum (FIG. 5D). This result established that the AP amplification loop was largely intact in properdin^{-/-} mice.

### Example 6

### Properdin and AP Play a More Significant Role in LOS- than in LPS-Induced Complement Activation In Vivo

Human properdin-deficient individuals are susceptible to lethal meningococcal is infection. Because N. meningitides bacteria contain lipooligosachamide (LOS) rather than LPS in their outer membranes, the role of properdin in N. meningitides LOS-induced complement activation was examined in vitro and in vivo. Using LOS-coated plate assays in Mg⁺⁺-EGTA, it was found that LOS, like LPS, induced AP complement activation in WT but not properdin^{-/-} mouse serum. Furthermore, by measuring plasma levels of activated C3, it was found that injection of LOS caused systemic complement activation in WT but not properdin^{-/-} mice in vivo (FIG. 6A). Notably, it was observed that LPS-induced systemic complement activation in vivo was reduced but not abolished in properdin^{-/-} mice (FIG. 6B). These results suggested that LOS activated complement in vivo principally via the AP pathway, whereas LPS activated complement through both AP-dependent and -independent pathways. Indeed, by performing LPS- or LOS-coated plate assays in GVB⁺⁺ buffer to enable all three complement activation pathways, it was demonstrated that fB or properdin deficiency caused a much more dramatic reduction in LOS-induced complement activation than in LPS-induced complement activation (FIG. 6C, D).

### Example 7

### Properdin is not Required for the AP Amplification Loop of the Classical Pathway Complement

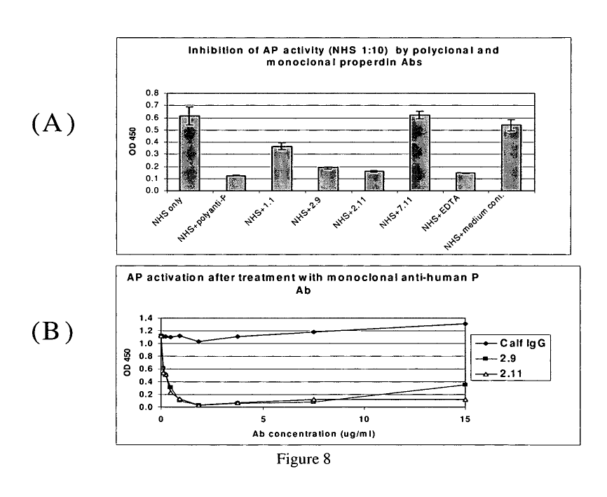

Monoclonal antibodies against human properdin were generated. The data in FIG. 8 to FIG. 14 demonstrate that anti-properdin mAbs selectively inhibit AP complement activation but have no effect on the AP amplification loop of the classical pathway complement. These properties of the antibodies make them distinct from the previously disclosed anti-human properdin antibodies which inhibited both the AP pathway complement and the classical pathway complement.

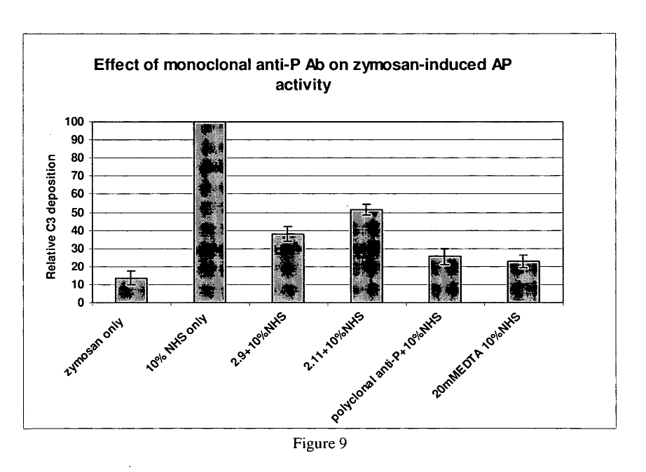

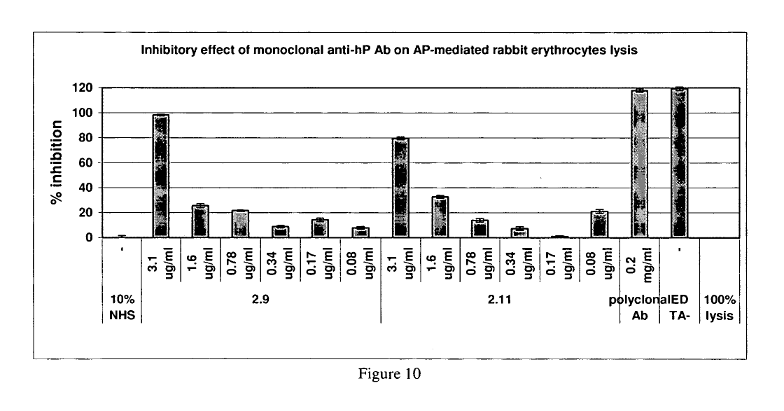

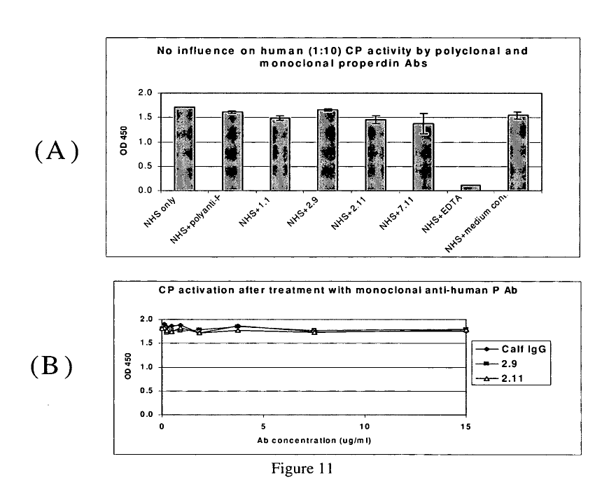

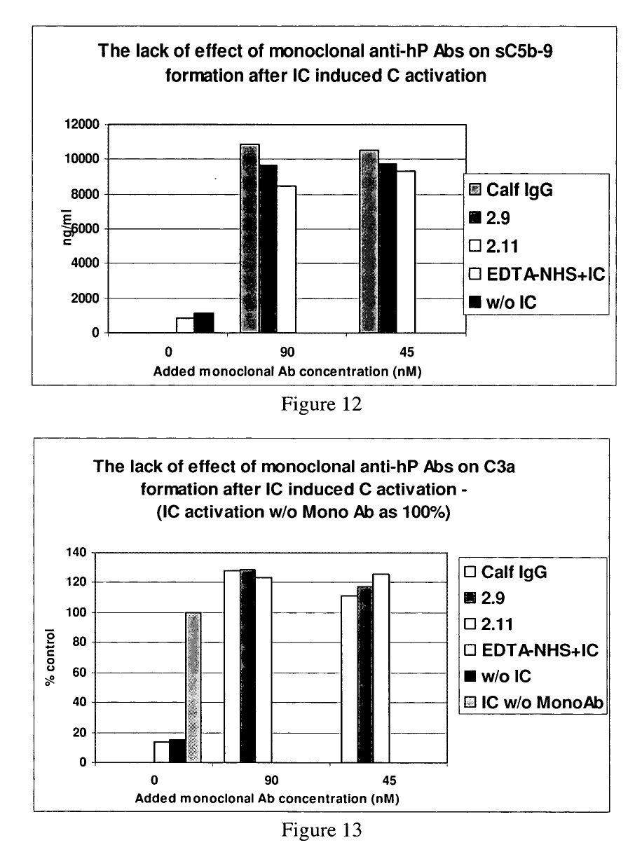

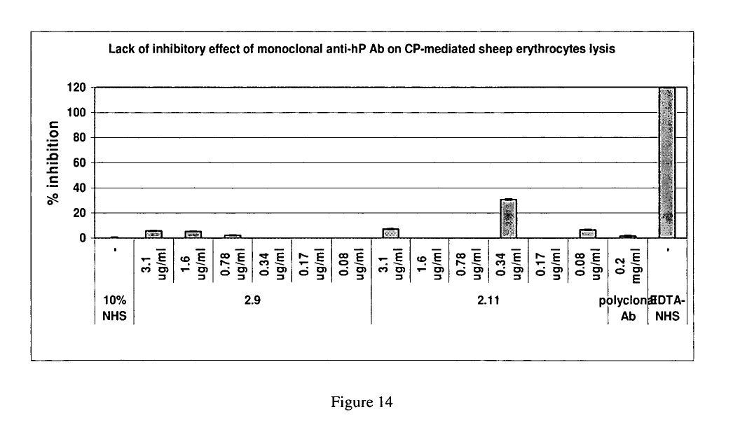

In FIG. 8 to FIG. 10, three different AP complement assays were used to demonstrate the inhibitory effect of anti-properdin mAbs. These assays are: LPS-induced AP complement activation (FIG. 8); zymosan-induced AP complement activation (FIG. 9); and rabbit erythrocyte-induced AP complement activation (FIG. 10). In FIG. 11 to FIG. 14, three different classical pathway complement activation assays were used to demonstrate the lack of effect of anti-properdin mAbs on the AP amplification loop of the classical pathway. These assays are: plate-bound OVA/anti-OVA immune complex-induced classical pathway complement activation (measured by plate C3 deposition, FIG. 11); OVA/anti-OVA immune complex-induced classical pathway complement activation in the fluid phase (measured by release of sC5b-9 and C3a, FIGS. 12 and 13); and antibody sensitized sheep erythrocytes-induced classical pathway complement activation (FIG. 14).

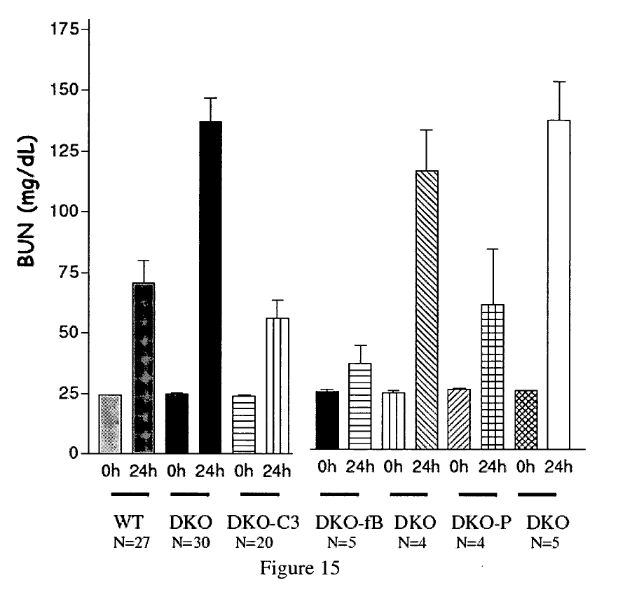

FIG. 15, show using properdin knockout mice to demonstrate that AP complement and properdin is critical in renal ischemia reperfusion injury.

Thus, anti-properdin reagents (mAbs, small molecule inhibitors etc) were developed for therapy in ischemia reperfusion injury.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method of treating an AP complement-mediated pathology in a subject, comprising the step of administering to said subject an alternative-pathway-specific anti-Properdin antibody, thereby inhibiting the generation of a C3bBb protein.

2. The method of claim 2, whereby the pathology is macular degeneration, ischemia reperfusion injury, arthritis, paroxysmal nocturnal hemoglobinuria (PNH) syndrome, atypical hemolytic uremic (aHUS) syndrome, asthma, organ transplantation sepsis, or their combination.

3. A method of inhibiting properdin-dependent, microbial antigen-, non-biological foreign surface- or altered self tissue-triggered AP complement activation in a subject, comprising the step of administering to said subject an alternative-pathway-specific, anti-Properdin antibody, thereby inhibiting the generation of a C3bBb protein.

4. The method of claim 3, whereby the AP complement activation results from recognition by said pattern recognition receptor of a microbial antigen selected from muramyl di-peptide (MDP), a CpG motif from bacterial DNA, peptidoglycan, lipoteichoic acid, outer surface protein A from Borrelia burgdorferi, synthetic mycoplasmal macrophage-activating lipoprotein-2, tripalmitoyl-cysteinyl-seryl-(lysyl)3-lysine (P3CSK4), dipalmitoyl-CSK4 (P2-CSK4), monopalmitoyl-CSK4 (PCSK4), amphotericin B, and a triacylated diacylated bacterial polypeptide, doubled stranded viral RNAs, blood tubing in cardio-pulmonary bypass surgery and kidney dialysis, apoptotic, necrotic and ischemia-stressed self tissues and cells, or their combination.

5. The method of claim 3, wherein the antibody does not affect the AP amplification loop of the classical pathway complement.

6. A transgenic non-human mammal and progeny thereof whose genome comprises a disruption of a Properdin-encoding gene such that the mammal lacks or has reduced levels of functional Properdin.

7. The transgenic non-human mammal of claim 6, wherein a neomycine cassette (NEO) is inserted between exons 5 and 6 of said Properdin gene.

8. The transgenic non-human mammal of claim 7, wherein the NEO results in the disruption of an intron between exons 5 and 6.

9. The transgenic non-human mammal of claim 6, wherein said transgenic mouse exhibits, relative to a wild-type mouse, a decreased activation of AP-compliment.

10. The transgenic non-human mammal of claim 6, wherein said transgenic mouse is fertile and transmits said transgene to its offspring.

11. A cell, organ, tissue or their combination, obtained from the transgenic non-human mammal of claim 6.

12. A method for identifying in vivo a biological activity of a compound, said method comprising the steps of:
a. providing a transgenic non-human mammal incapable of expressing properdin;
b. administering said compound to said non-human mammal;
c. determining an expressed pathology of said non-human mammal; and
d. identifying an in vivo biological activity of said compound.

13. The method of claim 12, wherein said biological activity is AP-compliment activation.

14. The method of claim 13, whereby the expressed pathology of said non-human mammal is macular degeneration, ischemia reperfusion injury, arthritis, paroxysmal nocturnal hemoglobinuria (PNH) syndrome, atypical hemolytic uremic (aHUS) syndrome, sepsis, bacterial lipooligosachamide (LOS) infection, or their combination.

15. A composition comprising the compound identified by the method of claim 14.

16. A method of treating an AP complement-mediated pathology in a subject, comprising the step of administering to said subject the composition of claim 15.

17. A method of making a transgenic non-human mammal comprising:
a. introducing into an embryo of the non-human mammal, a polynucleotide comprising a coding region for a disrupted intron of a Properdin-encoding gene;
b. transferring the embryo into a foster mother mouse;
c. permitting the embryo to gestate; and
d. selecting a transgenic mouse born to said foster mother mouse,
wherein said transgenic non-human mammal is characterized in that it has a decreased activation of AP-compliment when compared to a non-transgenic mammal.

18. The method of claim 17, wherein step of selecting comprises mating two selected transgenic mice; permitting the embryos to gestate; and selecting a transgenic mouse born to a transgenic mother.

19. The method of claim 18, wherein the method is repeated for more than one generation.

20. A method of culturing transgenic cells comprising the steps of:
a. providing the cell of claim 11; and
b. culturing said cell under conditions that allow growth of said cell.


SEQUENCE LISTING (12 sequences)

SEQ ID NO: 1 - DNA, 27 bp, murine
ctcgagcattcatctttgccagaaatc

SEQ ID NO: 2 - DNA, 21 bp, murine
tccccatactcagcactattg

SEQ ID NO: 3 - DNA, 34 bp, murine
ataacttcgtataatgtatgctatacgaagttat

SEQ ID NO: 4 - DNA, 61 bp, murine
gatatcataacttcgtataatgtatgctatacgaagttatgttcaatcacccaccatccct

SEQ ID NO: 5 - DNA, 27 bp, murine
ctcgagcattcatctttgccagaaatc

SEQ ID NO: 6 - DNA, 28 bp, murine
gcggccgcattccggctgtatctgagtc

SEQ ID NO: 7 - DNA, 27 bp, murine
gatatcaggaagaagtgaatatacagg

SEQ ID NO: 8 - DNA, 21 bp, murine
gggtgggattagataaatgcc

SEQ ID NO: 9 - DNA, 22 bp, murine
caaggtacggctttgttacaca

SEQ ID NO: 10 - DNA, 34 bp, murine; identical to SEQ ID NO: 3

SEQ ID NO: 11 - DNA, 23 bp, murine
cactgatattgtaagtagtttgc

SEQ ID NO: 12 - DNA, 22 bp, murine
ctagtgcgaagtagtgatcagg